(19) United States
(12) Patent Application Publication
(10) Pub. No.: US 2010/0121410 A1
(43) Pub. Date: May 13, 2010

(54) BIOMECHANICAL-STIMULATION APPARATUS AND METHOD FOR BONE REGENERATION

(75) Inventors: Joaquin Carlos de Larreta-Azelain Oliveras, Donostia-San Sebastian (ES); Jose Inaki Alava Marquinez, Donostia-San Sebastian (ES); Francisco Jose Estensoro Astigarraga, Donostia-San Sebastian (ES)

Correspondence Address: KNOBBE MARTENS OLSON & BEAR LLP, 2040 MAIN STREET, FOURTEENTH FLOOR, IRVINE, CA, 92614, US

(73) Assignee: OSETECH, S.L., E-20009 Donostia-San Sebastian (ES)

(21) Appl. No.: 12/376,274

(22) PCT Filed: Aug. 4, 2006
(86) PCT No.: PCT/ES06/00463

§ 371 Date: Nov. 12, 2009

(51) Int. Cl. A61N 1/00

(52) U.S. Cl. 607/52; 607/51

(57) ABSTRACT

The invention relates to a biomechanical stimulation apparatus for bone regeneration which comprises

at least one displaceable element (101) configured to be in contact with at least one part of a body of a living being, to exert a mechanical stimulus on said part of the body; and

displacement means (103-106) configured to displace said at least one displaceable element (101).

The apparatus is configured to displace said at least one displaceable element (101) such that said at least one displaceable element performs a movement according to a biometric wave.

The invention also relates to a biomechanical stimulation method and to a method for programming the apparatus.

### TECHNICAL FIELD OF THE INVENTION

The invention is comprised in the field of apparatuses, systems and methods for bone regeneration.

### BACKGROUND OF THE INVENTION

There are many methods for treating bone diseases, for example, osteoporosis: many pharmacological treatments are known which, however, can have problematic side-effects. There are also “natural” treatments without side-effects but with doubtful efficacy. Electromagnetic treatments in different forms and frequencies (see, for example, WO-A-2004/089467 and U.S. Pat. No. 6,321,119), surgical, laser and piezoelectric treatments (EP-A-0821929) are also known. In addition, treatments based on mechanical stimulation by means of ultrasonic (US-A-2001/027278) and mechanical (U.S. Pat. No. 5,376,065, ES-A-2155451, WO-A-2004/043324) systems are also known.

It is well known since 1981 (Woo, et al, “The Effect of Prolonged Physical Training on the Properties of Long Bone: a Study of Wolff's Law”, J Bone Joint Surg Am., June 1981, 63(5):780-7) that prolonged physical exercise and training have a beneficial effect on long bone maintenance and regeneration. In 1989, Alan A. Halpern proposed a system of vertical drops from a rigid platform as a means for alleviating low bone density and for improving bone system tone, without having to engage in intense physical exercise (U.S. Pat. No. 4,858,598). Soon afterwards, the company Osteo-Dyne, Inc. patented equipment for treating bone disorders, based on the mechanical compression of the patient by means of a continuous impact, which as a result of the piezoelectric properties of human bone generates electric signals which can stimulate bone growth (U.S. Pat. No. 5,484,388). However, these treatments characterized by strong impacts and high frequencies (of the order of 5 Hz or above can be difficult to maintain or even dangerous in elderly people with low bone density, furthermore not complying with standard ISO 2631 on the tolerance of vibrations on human beings, therefore their therapeutic application may be unadvisable.

In 1998, J. Flieger (J. Flieger, et al., “Mechanical Stimulation in the Form of Vibration Prevents Postmenopausal Bone Loss in Ovariectomized Rats”, Calcified Tissue International (publisher: Springer New York), Vol. 63, No. 6, pg. 510-514) proved that mechanical stimulation in the form of vibration prevents bone density loss in rats. In addition, C. Rubin et al. continue to develop the prevention of bone loss by high-frequency and low-magnitude mechanical stimuli, giving rise to many patents and patent applications of stimulation equipment based on vibration (U.S. Pat. No. 5,376,065, ES-2155451—corresponding to EP-B-0642331—, WO-A-2004/043324, JP-A-2004-147908, AU-B-2002300149, AT306969T, DE69827860T and WO-A-2005/115298). The basic idea of all this equipment is that a sinusoidal vibration wave, normally with a high frequency (of the order of 10-100 Hz) and with a very small displacement (0.01-2.0 mm), can stimulate bone regeneration and growth. However, these “hyperphysiological” frequencies are very far from the fundamental and primary harmonic frequencies applied in the bone by natural processes, such as those induced by walking or running.

Spanish utility model ES-U-1041026 describes a therapeutic vibrator which applies on the feet of a person several blows produced on a platform by means of cams having a special shape. This device attempts to transmit to the user vibrations “similar” to those occurring while walking or running.

ES-B1-2178971 describes a therapeutic system for the prevention, treatment and recovery of bone diseases based on periodic forces with a lower frequency than the previously described impulses.

### DESCRIPTION OF THE INVENTION

The present invention is based on the most natural method for bone regeneration, namely the relationship between physical exercise and the stimulation of the cells controlling bone formation.

A first aspect of the invention relates to a biomechanical stimulation apparatus for bone regeneration, comprising:

at least one displaceable element configured to be in contact with at least one part of a body of a living being (for example, with a foot), to exert a mechanical stimulus on said part of the body; and

displacement means configured to displace said at least one displaceable element.

According to the invention, the apparatus is configured to displace said at least one displaceable element such that said at least one displaceable element performs a movement according to a biometric wave.

This biometric wave can be a wave derived or derivable from a movement of at least one living being (for example, a human being or a group of human beings). The displaceable element can transfer to the living being any type of acceleration profile obtained from a natural movement of a living being, such as walking, running, jumping or jumping on tiptoe. What is transferred to the living being can be displacements or amplitudes, after a double integration of a previously obtained acceleration profile.

The movement can be, for example, a walking movement, a running movement, a jumping movement or a movement generated by a being standing on tiptoe and letting itself fall.

The biometric wave can be obtained or obtainable by means of a sensor (for example, an accelerometer or a pressure sensor) connected to the body of the living being, for example, to a limb (for example, to the ankle) of the being. If the acceleration is measured, position or displacement values can be obtained for the displaceable element by means of a double integration of the acceleration curve.

The apparatus can be configured and the displaceable element can be arranged such that the displaceable element is displaced according to a displacement pattern obtained or obtainable by means of a double integration of the biometric wave (i.e., for example, a biometric acceleration wave obtained by means of measurement on a live body, would pass to a distance, position and/or displacement wave which could be applied to control the displacement of the displaceable element).

Logically, a wave measured or a mean of a plurality of waves measured on the same person (or another type of living being), or on a plurality of persons (or other living beings) can be taken as a basis.

The apparatus can be configured to displace said at least one displaceable element such that it performs a movement with a repletion frequency between 0.1 and 1 Hz and with an amplitude between 5 and 70 mm. This movement can include at least one phase of acceleration between 1 and 3 g. The movement can be configured to cause between 10 and 50 microstrains. The term microstrains relates (at least in this document) to a measurement of the strains of a body, expressing the percentage of the total volume, measured in a strain direction. 10 microstrains therefore involve a strain of 10/1,000,000 times the length of the bone in the strain direction, and 50 microstrains involve a strain of 50/1,000,000 times said length.

The apparatus can be configured to make, during the operation of the apparatus, pauses between successive movement cycles of the displaceable element, said pauses lasting between 0.1 second and 1 second.

The apparatus can further comprise an electronic control system, the displacement means being configured to displace said at least one displaceable element under the control of said electronic control system, the electronic control system being configured to cause, through the displacement means, the displacement of the displaceable element according to said biometric wave.

The electronic control system can comprise at least one memory in which data relating to said biometric wave is stored. For example, data of a plurality of biometric waves corresponding to persons with different characteristics can be stored in at least one memory of the apparatus, the apparatus further comprising selection means configured such that the displaceable element can be displaced according to a biometric wave selected from said plurality of biometric waves. A “library” of biometric waves (for example, organized according to age, weight and/or sex, etc.) can thus be available, from which the most suitable wave for a specific person can be selected, without having to carry out measurements on said person to obtain his or her specific “biometric wave”. This biometric wave selection can be carried out manually, for example, by means of a keyboard associated to the apparatus or to a command or control device outside the apparatus (for example, a remote control). The biometric wave considered as the “most suitable” wave according to the specific characteristics of a person (for example, according to his or her age, sex, height, weight, etc.) can thus be chosen without having to carry out measurements on said person.

The biometric wave can alternatively or complementarily correspond to a mean of biometric waves obtained by means of measurements carried out on a plurality of different persons.

The apparatus can additionally comprise means for receiving a signal from an external sensor (for example, an acceleration or pressure sensor) (for example, attached to a limb of a person who is subjected to a treatment with the apparatus) and means for modifying at least one aspect of the operation of the apparatus according to said signal. The displacements on the person who is subjected to the treatment can thus be measured and the operation of the apparatus can be adapted so that the displacements “received” and “felt” by the person are optimally adjusted to the biometric wave to be applied. This can be carried out with software configured to minimize the displacement detected by the sensor and the “desired” displacement data stored in the memory of the electronic control system.

The displaceable element can be configured so that a person can stand on his or her feet on said displaceable element. The elements can also be configured to act on other parts of the body, and even to treat feet or other parts of the body from other angles or directions. For example, in the case of applications for microgravity environments (for example, in a spacecraft or space station), the apparatus can be configured to be “coupled” to the person and secure him or her, to prevent him or her from being displaced as a result of the displacements.

Each displaceable element can be pivotably arranged about a shaft, to “simulate” a walking movement.

Another aspect of the invention relates to a biomechanical stimulation method for bone regeneration of a living being, comprising the step of repetitively generating a displacement on an object (for example, a sole of a foot) associated to a bone structure, in order to mechanically stimulate said bone structure. According to the invention, the displacement is generated according to a biometric wave, for example, a wave derived or derivable from a movement of a living being.

That stated in relation to the apparatus is also applicable to the method, mutatis mutandis.

For example, the living being can be a human being.

The movement can, for example, be a walking movement, a running movement, a jumping movement or a movement generated by a being standing on tiptoe and letting itself fall.

The biometric wave can be obtained or obtainable by means of a sensor connected to the body of a living being; the sensor can be, for example, an accelerometer or a pressure sensor. The sensor can, for example, be connected to a limb of the being (for example, to its ankle).

The displacement can be generated according to a displacement pattern obtained by means of a double integration of the biometric wave. In other words, for example, a biometric acceleration wave obtained by means of a measurement on the living being would pass to a distance, position and/or displacement wave which would directly guide the displacement of the object associated to the bone structure.

The displacement can, for example, be carried out with a repetition frequency between 0.1 and 1 Hz and with a movement with an amplitude between 5 and 70 mm, and the movement can optionally include at least one phase of acceleration between 1 and 3 g.

The movement can be configured to cause between 10 and 50 microstrains.

Pauses can be made between successive movement cycle, said pauses being, for example, between 0.1 second and 1 second.

The displacements can be generated under the control of an electronic control system acting on displacement means configured to displace the at least one displaceable element to generate the displacements.

According to a possible embodiment of the invention, the biometric wave can be selected from a plurality of stored biometric waves, according to at least one characteristic of the living being to which the biomechanical stimulation is to be applied. In other words, a “library” of stored biometric waves (organized by characteristics such as, for example, age, weight, heights and/or sex, etc.) can thus be available, and the most suitable biometric wave for a specific person according to the characteristics of said person can be selected, without having to carry out measurements on said specific person. This can be practical to reduce the work related to the treatment of a person, as the step of obtaining a specific biometric wave for said person, by means of measurements carried out on the person himself or herself, can be eliminated.

The method can be a method for stimulating a bone structure for experimental purposes.

The method can be a method for stimulating a bone structure of a human being.

According to a possible embodiment of the invention, a result of the displacement on the object can be measured to obtain data relating to at least one effect of said displacement, and in which said data is used to modify the way in which subsequent displacements on the object are generated. In other words, it is a “feedback” system for adjusting the parameters of the displacements generated so that the “received” displacements are adjusted to the desired characteristics (i.e., to the biometric wave).

Another aspect relates to a method for programming an apparatus according to that described above, and comprising the steps of obtaining a signal from a movement of a living being, and programming an electronic control system of the apparatus with said signal or with a signal derived from said signal, such that the apparatus displaces a displaceable element according to a biometric wave associated to said signal. The signal which is obtained from the living being can be a signal indicating an acceleration of a part of the body of the living being, and said signal can be successively integrated to obtain a signal indicating position or displacement.

It can therefore be stated that the invention intends to generate a mechanical stimulation based on what actually occurs when a natural movement is performed (for example, walking, running or jumping).

### DESCRIPTION OF THE DRAWINGS

To complement the description and with the aim of aiding to better understand the features of the invention according to preferred practical embodiments thereof, a set of drawings is attached as an integral part of the description in which the following has been shown with an illustrative and non-limiting character:

FIG. 1 shows a biometric wave corresponding to the acceleration of the ankle of a person while walking.

FIG. 2 shows a biometric velocity wave obtained by means of integrating the wave shown in FIG. 1.

FIG. 3 shows a biometric position or displacement wave obtained by means of integrating the wave shown in FIG. 2.

FIG. 4 shows an experimental configuration for stimulating a bone matrix.

FIGS. 5 and 6 show experimental data obtained.

FIGS. 7 and 8 show photographs of the matrix structure; FIG. 7 corresponds to the situation after 7 days without stimulus, and FIG. 8 to the situation after 7 days with stimulus.

FIG. 9 schematically shows an accelerometric system used to determine natural movement wave patterns.

FIGS. 10A-10D show an elevational longitudinal section view, a bottom plan view, a cross-section view and a perspective view, respectively, of a mechanism implementing the electromechanical part of an apparatus according to a possible embodiment of the invention.

FIG. 11 schematically shows the main functional components of an apparatus according to a preferred embodiment of the invention.

FIGS. 12A-12F show acceleration curves similar to FIG. 1, for 6 different persons.

FIGS. 13A-13F show the same as FIGS. 12A-12F, respectively, but for the running movement.

FIGS. 14A-14F show the same as FIGS. 12A-12F, respectively, but for the jumping movement.

FIGS. 15A-15F show the same as FIGS. 12A-12F, respectively, but for the movement of standing on tiptoe and letting oneself fall.

### PREFERRED EMBODIMENT OF THE INVENTION

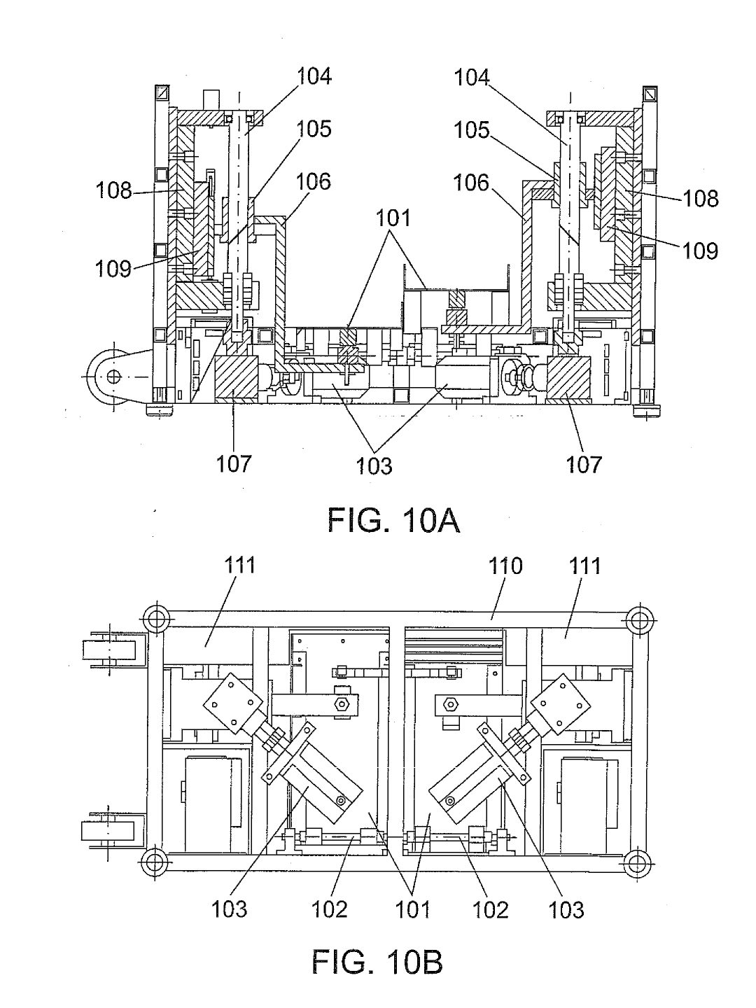

Acceleration characteristics of several natural movements (walking, running or jumping), as schematically shown in FIG. 9, have been analyzed and defined by using an accelerometric system coupled in the foot at the height of the ankle and a data recording and processing system (in this case including Measurements Studio® and Matlab®), on a human population with a different physical profile (gender, height, weight). The movement of the legs has been monitored by placing an acceleration sensor 91 in the right foot at the height of the ankle (in this case, in the inner part of the leg), which detects the accelerations in the x (vertical) and y (horizontal) axes. In this case, since biometric waves or curves intended to be applied in a machine which will vertically stimulate the sole of the feet are obtained, the information which has been sought relates to the x axis. The acceleration data has been captured and stored by means of the Measurement Studio® software 92 of National Instruments®, whereas graphs have been subsequently processed and obtained with Matlab®.

It has been verified that the waveforms for each movement are similar, mainly varying in intensity. It has then been verified that said wave can stimulate osteoblast metabolism and growth by using a simulation system with cell culture supports and human osteoblasts.

Biometric studies have been conducted on the acceleration curves or waves corresponding to the movement of a person while walking, running, jumping, standing on tiptoe, letting himself or herself fall. It is possibly especially suitable to start from the movement corresponding to walking (bone cells will thus be excited with the same accelerations undergone by the ankle of a person while walking). This is caused by the fact that walking is the predominant exercise in human beings and is therefore more usual, from the point of view of cell growth and activation, than running, jumping or standing on tiptoe to subsequently let oneself fall, which are more violent movements. Furthermore, an elderly person can walk but can have difficulty running or jumping.

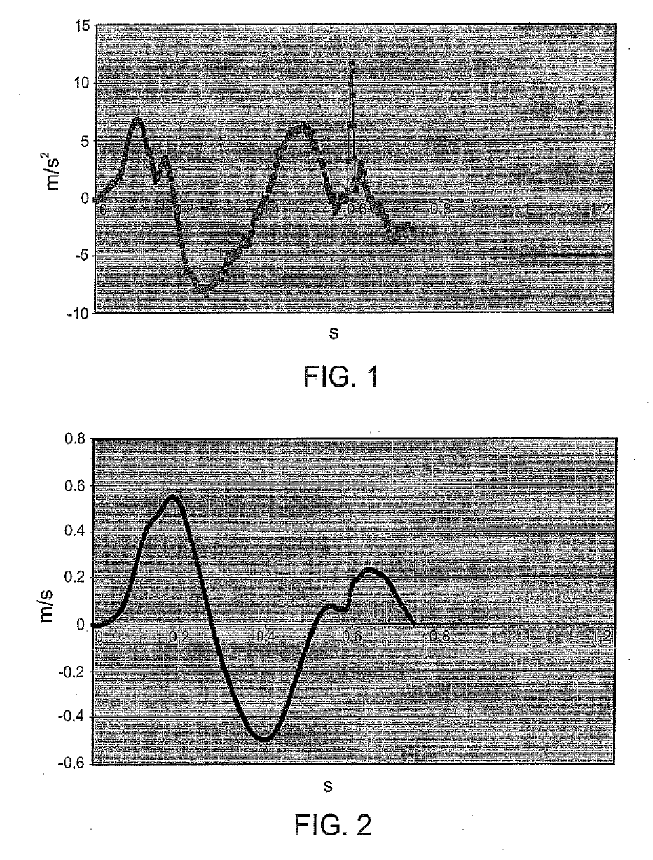

FIG. 1 (vertical axis: acceleration in m/s²; horizontal axis: time in seconds) shows the acceleration measured in the ankle in a person (a woman in this specific case). In other words, the curve shows the acceleration of the ankle of the woman while walking.

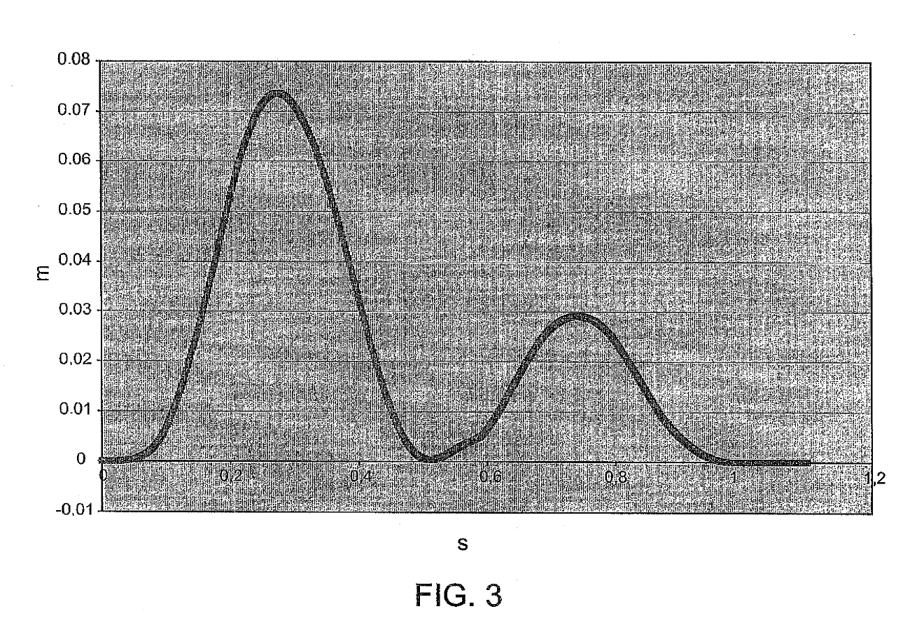

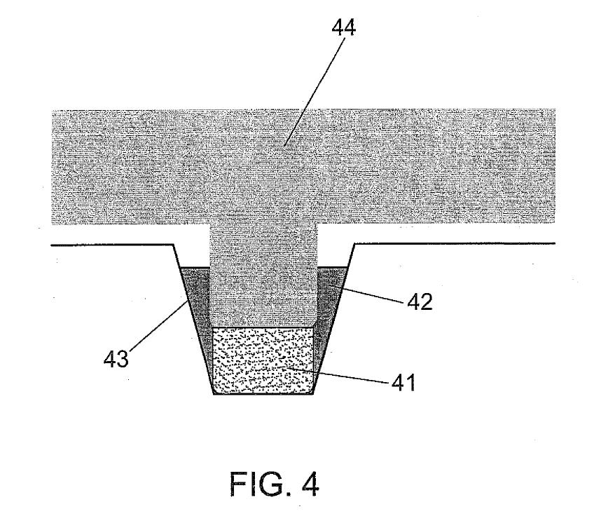

FIG. 2 shows a signal obtained by means of integrating the curve shown in FIG. 1; the vertical axis represents the velocity (m/s) and the horizontal axis shows the time (in s). It has been considered that a suitable stimulation can be carried out by means of an element which is displaced according to this velocity profile. To that end, the velocity curve can be integrated and a curve relating the time with a certain amplitude or displacement of a displaceable element can thus be obtained; thus, by means of a conventional displacement control system an apparatus can be programmed so that it displaces a displaceable element such that it adopts at each time a position (for example, a height) according to said displacement curve. FIG. 3 (vertical axis: displacement in meters (m); horizontal axis: time in seconds (s)) shows the displacement curve obtained by means of integrating the velocity curve of FIG. 2.

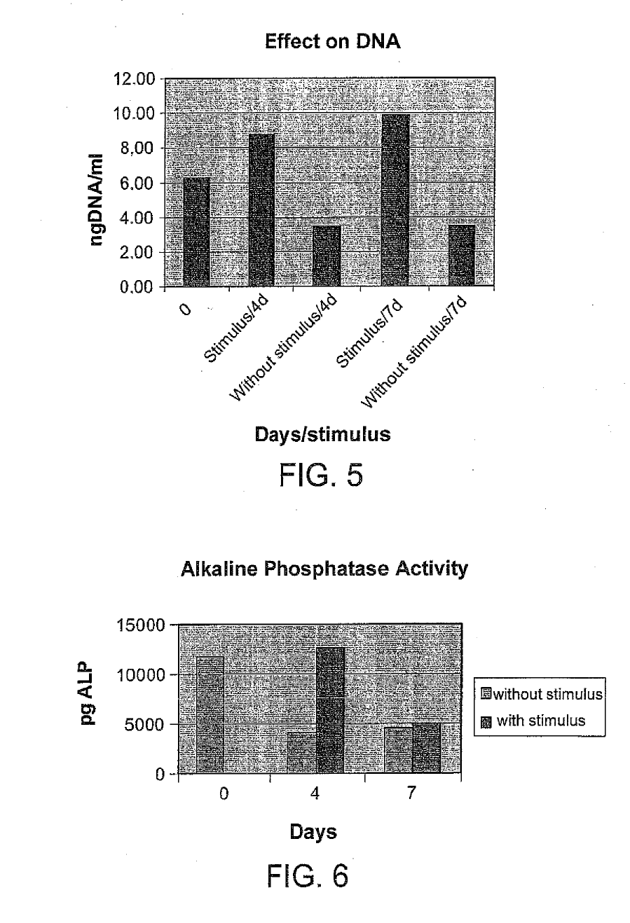

As an example of the stimulation effect of the wave shown in FIG. 3, said wave has been applied, as a mechanical stimulus, to a culture of bone cells (osteoblasts) located in a calcium phosphate matrix 41 (Beckton & Dickinson brand commercial matrix) simulating the bone (see FIG. 4). The intention was to this compare the results obtained by applying the wave, with the results obtained in the event that no stimulus is applied. To that end, calcium phosphate matrices for cell culture, inside a 96-well plastic plate, and a movement simulator which can reproduce the biometric wave have been used. The matrices 41 were kept secured inside the plate with a silicone buffer.

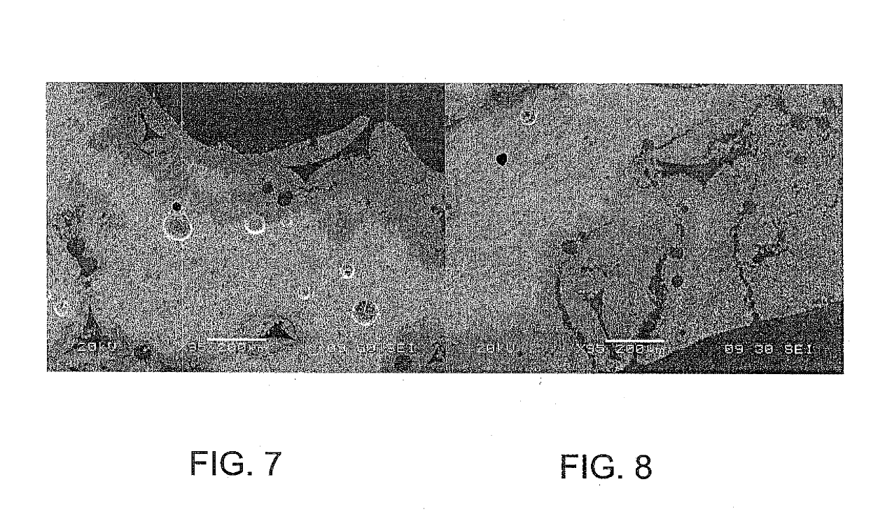

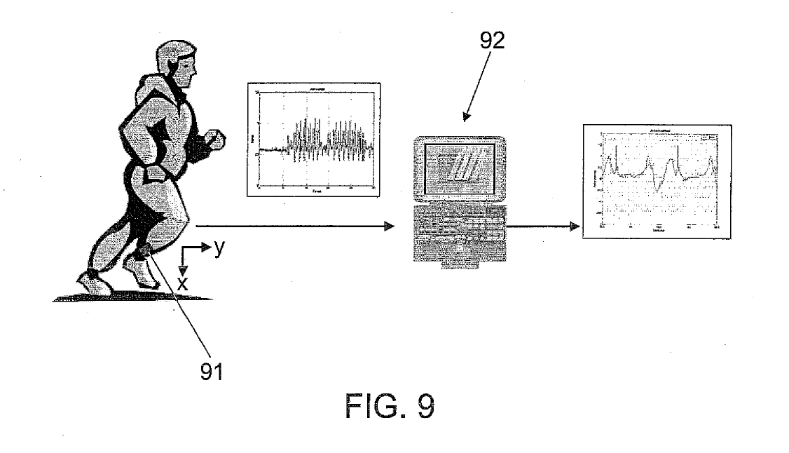

The simulation matrices 41 were seeded with 5×10⁵ cells from the ATCC cell line CRL-11372, and were incubated under stirring at 37° C. for 6 hours, followed by centrifugation (5 minutes, 14500 rpm). The matrices thus seeded were carefully placed with tweezers in the definitive assay wells 43, adding 250 μl of fresh culture medium 42. The seeded matrices were kept in normal culture for 24 hours to allow the establishment of a minimum initial population. Every morning, from day zero onwards, the culture medium was removed from the well and 120 μl of fresco culture medium (enough to cover the matrix) were added. It was then covered with a silicone membrane 44 and pressure-fitted in a stimulation apparatus which was in turn introduced in a CO₂ oven. From this moment, a computer-generated program for stimulating by means of the biometric wave was activated, for 5 hours every day, with the oven closed at 37° C. After the 5 hours, the plate was removed from the stimulation equipment/oven and the cover with membrane was again removed under a hood. The old medium was eliminated and 250 μl of fresh culture medium were replaced. This was carried out for the 7 days that the assay lasted. Control samples were taken at the start and end of the assay. The alkaline phosphatase (ALP) activity (FIG. 6; the vertical axis of the diagram indicates the amount of alkaline phosphatase in picograms (pgALP)), the amount of DNA in the samples (FIG. 5) and the changes in the matrix structure (FIG. 7—showing the structure after 7 days without stimulation—and FIG. 8—showing the structure after 7 days with stimulation) were analyzed in each time period (0, 4, 7 days).

As can be observed, the capacity of the wave to stimulate human osteoblast growth and metabolic activity (see FIG. 8) considerably increase cell proliferation and activity (see FIGS. 5 and 6).

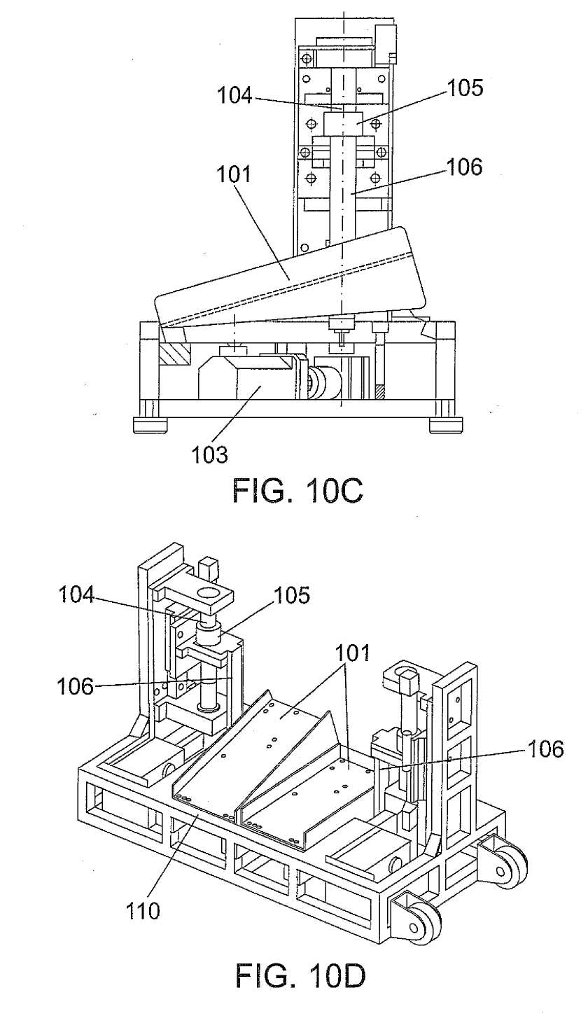

The application of the stimulation to a person can be carried out with a device or apparatus such as that shown in FIGS. 10A-10D, and comprising two platforms 101 each of which is pivotable about a shaft 102, in order to perform a pivoting or rocking movement imitating, to a certain extent, the movement caused by the foot while walking. It has been verified that this movement can be preferred because a purely linear movement of the platforms could give the person an unpleasant “jumping” feeling. When the treatment is applied to the person, he or she can stand on the machine, with a foot supported on each platform (other practical embodiments of the invention can be designed to apply a treatment to a person in a horizontal position or any other position; other embodiments of the invention can further be configured to apply a treatment to other areas of the body and not only to the feet).

The movement of the platforms 101 is induced with respective electric motors 103 which make respective threaded spindles 104 rotate, on which spindles respective nuts 105 linked to a support system 106 of the platforms are screwed. Thus, when the spindles 104 rotate in one direction or another, the corresponding nuts 105 move upwards and downwards and the corresponding upward or downward rocking of the platforms 101 occurs.

The movement is controlled by means of using “electronic cams” controlling the rotational speed of the motors and therefore the rotational speed of the spindles. By means of controlling (with a variator) the rotational speed of the motor, the displacements required in the nut of the spindle are achieved. Each support plate or platform 101 for supporting each foot can be moved independently and according to the same acceleration profile. The support plates or platforms for supporting the feet of the patient pivot on the shaft 102 at the front end of the platform, as has been indicated above.

The software for controlling the movement of the platforms can be developed by means of integrating the acceleration profile into velocity and displacement profiles. Several movement curves are programmed to the electronic cams with these profiles. Subsequently it is possible to validate the accelerations caused in the ankle of type persons, i.e., in persons showing different types of body constitutions. These validations can be used to ensure that the acceleration profile applied by the therapeutic machine is similar to that measured for a person while walking. To perform the validation, the acceleration in the ankle of a person applied during the operation of the machine can be measured by means of accelerometers and can be compared with the acceleration profile used to program the electronic cams.

The machine can comprise the following subassemblies and main components, some of which are shown in FIGS. 10A-10D:
Motor controllers /variators.
Motors 103.
Retransmissions 107.
Spindles 104.
Spindle support subassemblies 108.
Nuts 105 coupled on the spindles.
Nut anti-rotation guides 109.
Pivot shafts 102.
Platforms 101 for raising and supporting the person.
Subassemblies 106 for applying the movement of each spindle to the corresponding platform.
General structure and casing 110.
On and off controls.
Electric cupboard with safety devices according to regulations 111.

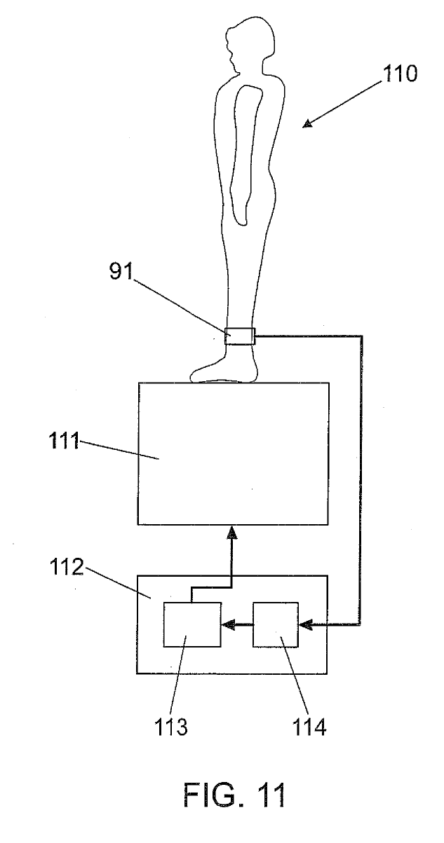

FIG. 11 schematically shows the machine according to a preferred embodiment thereof. A person 110 is located on an electromechanical part 111 of the machine, which can comprise a mechanism such as that shown in FIGS. 10A-10D, in which case the person can be standing, with each foot supported on one of the aforementioned platforms 101. In addition, the machine comprises an electronic control module or system 112 comprising electronic means 113 to make the motors of the electromechanical part (for example, the aforementioned motors 103) operate such that they displace the platforms according to the corresponding biometric wave, stored in a memory of said electronic means 113.

In addition and according to a possible embodiment of the invention, the machine can be configured to use a data feedback which allows ensuring that the user actually receives a displacement according to the corresponding biometric wave, and/or to “validate” the apparatus for type persons. The wave that the machine applied on the user is not as important as the wave that the user receives. To achieve a maximum coincidence between the wave to be received by the user and the wave that the user actually receives, a feedback system based on an accelerometer or sensor 91 (which can be identical or similar to that used to obtain the original biometric wave, as has been described in relation to FIG. 9) can be incorporated. This sensor is coupled to the user (for example, to his or her ankle) and the output signal of the sensor is received in the electronic control system 112 having calculation means 114 for determining a difference between the wave received by the user and the desired wave, and for modifying the operation of the machine to minimize this difference. The person skilled in the art can easily develop the suitable software according to the hardware used in this specific case, it is therefore not necessary to describe this aspect with more detail.

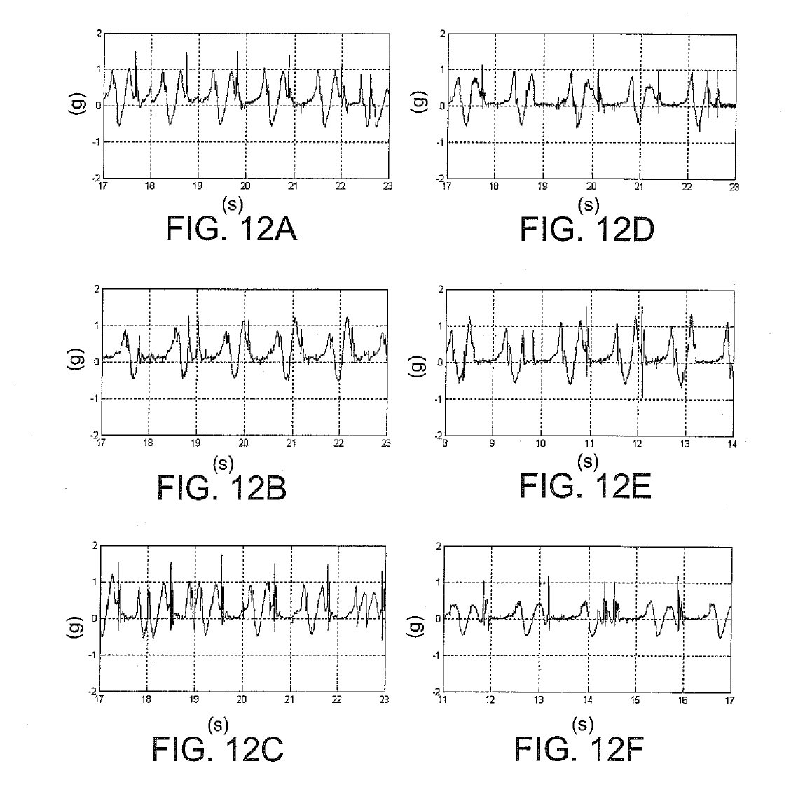

FIGS. 12A-12F show the acceleration curves similar to FIG. 1 for 6 different persons (FIGS. 12A-12C show the acceleration measured in the ankle for three different women and FIGS. 12D-12F show the acceleration measured in the ankle for three different men), while walking. As can be observed from the figures, the curves are quite different, i.e., the acceleration curve while walking varies among different persons.

To apply a suitable treatment to a person, it is possible to use for each person his or her own biometric wave (for example, a displacement curve obtained from a double integration of the acceleration measured on this same person), or use a biometric wave calculated from a mean of biometric waves measured on a plurality of persons (i.e., it would be a “typical” wave for a certain movement). It is also possible to have a “library” of biometric waves (for example, organized according to ages, weights, heights, sex, etc.), from which the most suitable wave for a specific person can be selected, without having to carry out measurements on said person.

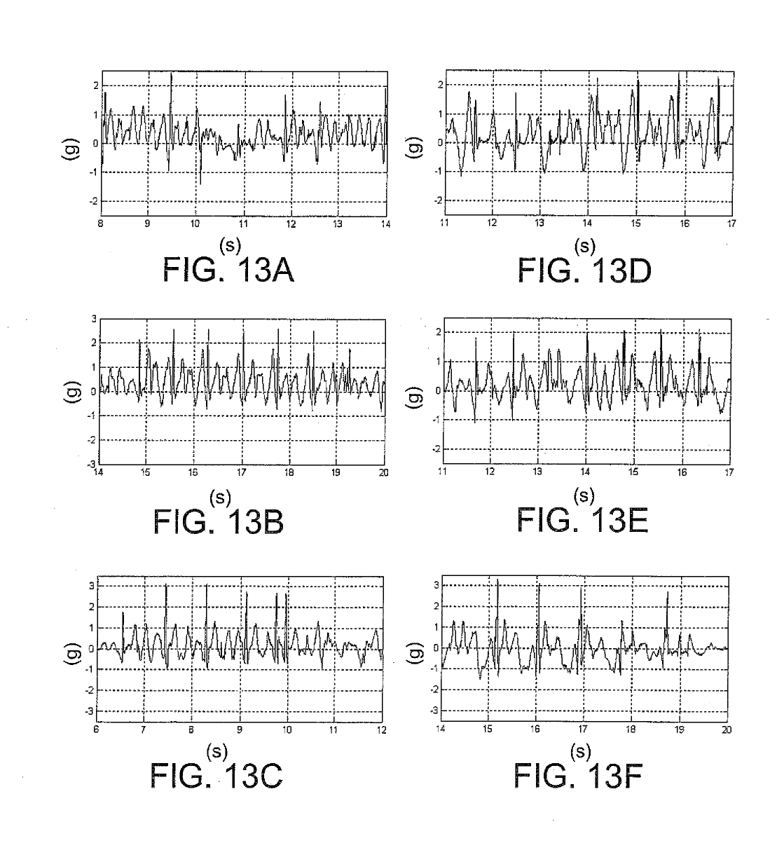

FIGS. 13A-13F show the same as FIGS. 12A-12F, respectively, but for the running movement.

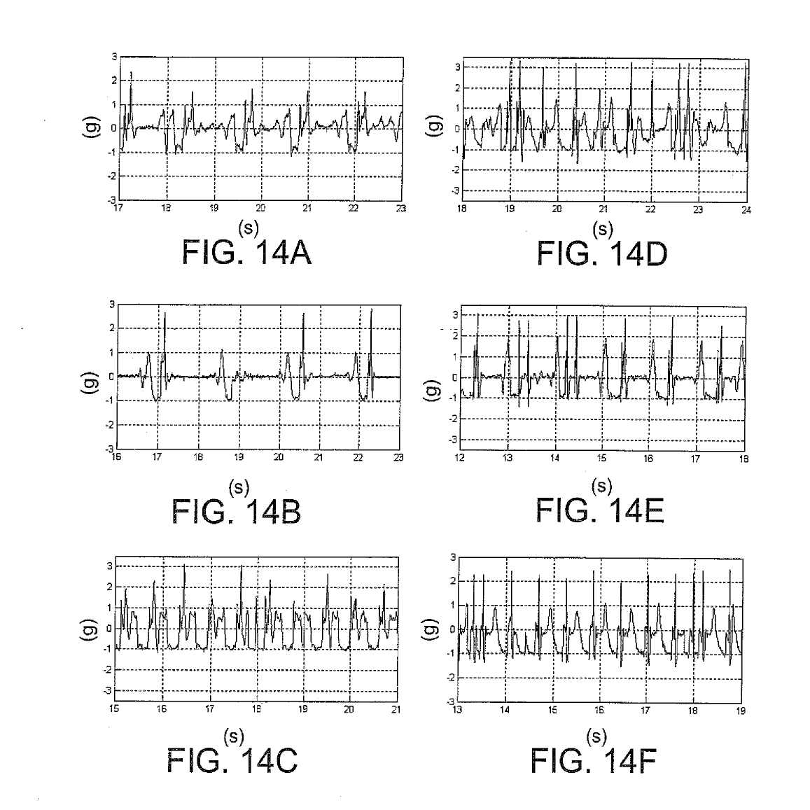

FIGS. 14A-14F show the same as FIGS. 12A-12F, respectively, but for the jumping movement.

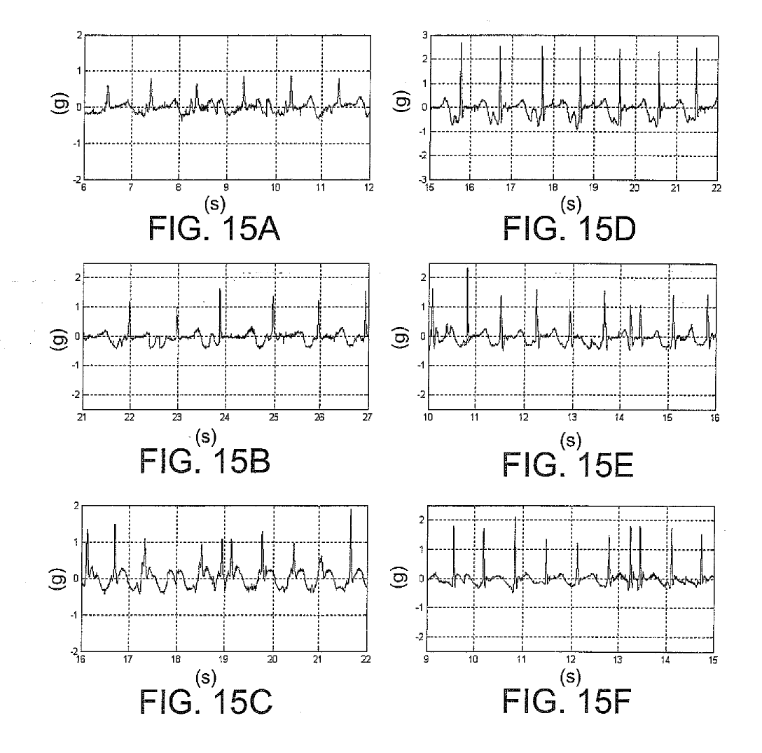

FIGS. 15A-15F show the same as FIGS. 12A-12F, respectively, but for the movement of standing on tiptoe and letting oneself fall.

It can be observed that the acceleration curves are very dependent on the type of movement being performed.

In this text, the word “comprises” and its variants (such as “comprising”, etc.) must not be interpreted in an exclusive manner, i.e., they do not exclude the possibility of that described including other elements, steps etc.

In addition, the invention is not limited to the specific embodiments which have been described but also covers, for example, the variants which have been carried out by the person skilled in the art (for example, as regards the choice of materials, dimensions, components, configuration, etc.), within that inferred from the claims.

1. A biomechanical stimulation apparatus for bone regeneration, comprising
at least one displaceable element configured to be in contact with at least one part of a body of a living being, to exert a mechanical stimulus on said part of the body; and
displacement means configured to displace said at least one displaceable element;
characterized in that
the apparatus is configured to displace said at least one displaceable element such that said at least one displaceable element performs a movement according to a biometric wave,
and in that
the apparatus is configured to make, during the operation of the apparatus, pauses between successive movement cycles of the displaceable element, said pauses lasting between 0.1 second and 1 second.

2. The apparatus according to claim 1, wherein the biometric wave is a wave derived from a movement of at least one living being.

3. The apparatus according to claim 1, wherein the biometric wave is a wave derivable from a movement of at least one living being.

4. The apparatus according to claim 1, wherein the living being is a human being.

5. The apparatus according to claim 2, wherein the movement is a walking movement.

6. The apparatus according to claim 2, wherein the movement is a running movement.

7. The apparatus according to claim 2, wherein the movement is a jumping movement.

8. The apparatus according to claim 2, wherein the movement is a movement generated by a being standing on tiptoe and letting itself fall.

9. The apparatus according to claim 1, wherein the biometric wave is obtained or obtainable by means of a sensor connected to the body of a living being.

10. The apparatus according to claim 9, wherein said sensor is an accelerometer or a pressure sensor.

11. The apparatus according to claim 9, wherein said sensor is connected to a lower limb of the being.

12. The apparatus according to claim 11, wherein said sensor is connected to an ankle of the being.

13. The apparatus according to claim 1, wherein the displaceable element is arranged to be displaced according to a displacement pattern obtained by means of a double integration of the biometric wave.

14. The apparatus according to claim 1, which is configured to displace said at least one displaceable element such that it performs a movement with a repetition frequency between 0.1 and 1 Hz and with an amplitude between 5 and 70 mm.

15. The apparatus according to claim 14, wherein said movement includes at least one phase of acceleration between 1 and 3 g.

16. The apparatus according to claim 14, wherein said movement is configured to cause between 10 and 50 microstrains.

17. The apparatus according to claim 1, further comprising an electronic control system, the displacement means being configured to displace said at least one displaceable element under the control of said electronic control system, the electronic control system being configured to cause, through the displacement means, the displacement of the displaceable element according to said biometric wave.

18. The apparatus according to claim 17, wherein the electronic control system comprises at least one memory in which data relating to said biometric wave is stored.

19. The apparatus according to claim 18, wherein data of a plurality of biometric waves corresponding to persons with different characteristics is stored in at least one memory of the apparatus, the apparatus further comprising selection means configured such that the displaceable element can be displaced according to a biometric wave selected from said plurality of biometric waves.

20. The apparatus according to claim 17, additionally comprising means for receiving a signal from an external sensor and means for modifying at least one aspect of the operation of the apparatus according to said signal.

21. The apparatus according to claim 1, characterized in that said displaceable element is configured so that a person can stand on his or her feet on said displaceable element.

22. The apparatus according to claim 1, comprising two of said displaceable elements, each one of said two displaceable elements comprising a platform configured to support a foot of the living being, each platform being pivotable about a shaft, in order to perform a pivoting movement in accordance with said biometric wave.

23. A biomechanical stimulation method for bone regeneration, comprising the step of repetitively generating a displacement on an object associated to a bone structure of a living being, in order to mechanically stimulate said bone structure, characterized in that
the displacement is generated according to a biometric wave,
and in that
pauses are made between successive movement cycles, said pauses being between 0.1 second and 1 second.

24. The method according to claim 23, wherein the biometric wave is a wave derived from a movement of at least one living being.

25. The method according to claim 23, wherein the biometric wave is a wave derivable from a movement of at least one living being.

26. The method according to claim 23, wherein the living being is a human being.

27. The method according to claim 24, wherein the movement is a walking movement.

28. The method according to claim 24, wherein the movement is a running movement.

29. The method according to claim 24, wherein the movement is a jumping movement.

30. The method according to claim 24, wherein the movement is a movement generated by a being standing on tiptoe and letting itself fall.

31. The method according to claim 23, wherein the biometric wave is obtained or obtainable by means of a sensor connected to the body of a living being.

32. The method according to claim 31, wherein said sensor is an accelerometer or a pressure sensor.

33. The method according to claim 31, wherein said sensor is connected to a lower limb of the being.

34. Method according to claim 33, wherein said sensor is connected to an ankle of the being.

35. The method according to claim 24, wherein the displacement is generated according to a displacement pattern obtained by means of a double integration of the biometric wave.

36. The method according to claim 23, wherein the displacement is carried out with a repetition frequency between 0.1 and 1 Hz and with a movement with an amplitude between 5 and 70 mm.

37. The method according to claim 36, wherein said movement includes at least one phase of acceleration between 1 and 3 g.

38. The method according to claim 36, wherein said movement is configured to cause between 10 and 50 microstrains.

39. The method according to claim 23, wherein the displacements are generated under the control of an electronic control system acting on displacement means configured to displace the at least one displaceable element to generate the displacements.

40. The method according to claim 23, wherein the biometric wave is selected from a plurality of stored biometric waves, according to at least one characteristic of the living being to which the biomechanical stimulation is to be applied.

41. The method according to claim 23, for stimulating a bone structure for experimental purpose.

42. The method according to claim 23, for stimulating a bone structure of a human being.

43. The method according to claim 23, wherein a result of the displacement on the object is measured to obtain data relating to at least one effect of said displacement, and wherein said data is used to modify the way in which subsequent displacements on the object are generated.

44. The method according to claim 23, comprising displacing two feet of the living being by means of two platforms configured to each support a foot of the living being, each platform being pivotable about a shaft, in order to perform a pivoting movement in accordance with said biometric wave.

45. A method for programming an apparatus according to claim 17, characterized in that it comprises the steps of obtaining a signal from a movement of a living being, and programming the electronic control system of the apparatus with said signal or with a signal derived from said signal, such that the apparatus displaces the displaceable element according to a biometric wave associated to said signal.

46. The method according to claim 45, wherein the signal which is obtained from the living being is a signal indicating an acceleration of a part of the living being, and in that said signal is integrated to obtain a signal indicating position or displacement.
